(19) United States
(12) Patent Application Publication
(10) Pub. No.: US 2022/0017571 A1
(43) Pub. Date: Jan. 20, 2022

(54) METHODS FOR CHROMATOGRAPHIC PROTEIN EXTRACTION AND PURIFICATION

(71) Applicant: Purolite (China) Co., Ltd., Zhejiang (CN)

(72) Inventors: Alessandra Basso, Udine (IT); Benjamin Summers, Pontyclun RCT (GB); Simona Serban, Cardiff (GB); Christopher Bresner, Cowbridge (GB)

(21) Appl. No.: 17/379,605

(22) Filed: Jul. 19, 2021

Related U.S. Application Data
(60) Provisional application No. 63/053,942, filed on Jul. 20, 2020.

(51) Int. Cl. C07K 1/18; C07K 14/415; B01J 39/26; B01J 39/19; B01D 15/36; B01D 15/16; B01D 15/42; A23L 5/20; A23L 33/185

(52) U.S. Cl. CPC C07K 1/18; C07K 14/415; B01J 39/26; B01J 39/19; A23V 2002/00; B01D 15/166; B01D 15/426; A23L 5/273; A23L 33/185; B01D 15/362

(57) ABSTRACT

Disclosed herein are methods and compositions for isolating a protein fraction from a potato sample. The methods include adjusting the potato sample containing the protein fraction to a pH of about 4.0 to 5.2; and loading the potato sample onto a sulfonated epoxy resin, wherein the sulfonated epoxy resin comprising Formula (I) is adjusted to a pH of about 4.0 to 4.5. The methods also include washing the sulfonated epoxy resin and eluting the protein fraction.

### CROSS-REFERENCE TO RELATED APPLICATIONS

This application claims priority to U.S. Provisional Patent Application No. 63/053,942, filed Jul. 20, 2020. The entire content of the foregoing application is incorporated herein by reference.

### TECHNICAL FIELD

This disclosure relates to methods of isolating proteins of interest using chromatographic protein extraction and purification methods that utilize an sulfonated epoxy resin that enhances isolation of protein (e.g., patatin) from a sample (e.g., potato juice).

### BACKGROUND

Fresh potato juice is a complex mixture of soluble and insoluble material. It has a high protein content, and further comprises residual starch, minerals, toxic glycoalkaloids, and monomeric and polymeric reactive phenols. It is a by-product of potato starch production. However, after potato starch production, potato juice often is disposed as waste. Potato juice contains a relatively high concentration of proteins, up to 1.5% by weight. The proteins included in potato juice generally can be divided into three groups: (i) a high molecular weight (HMW) fraction of highly homologous acidic 43 kDa glycoproteins (40-50 weight percentage of total potato protein), (ii) basic low molecular weight (LMW) 5-25 kDa among which are glycoproteins (30-40 weight percentage of total potato protein) and (iii) other proteins (10-20 weight percentage of total potato protein).

Patatin is a family of glycoproteins that has lipid acyl hydrolase and transferase activities and will predominantly be part of the HMW fraction of potato juice. A function of patatin involves its use as a storage protein. It, however, also has lipase activity and can cleave fatty acids from membrane lipids. (Shewry, Ann Bot (2003) 91(7):755-69; Banfalvi et al., Mol Gen Genet. (1994) 245(4):517-22). In fact, the patatin protein makes up about 40% of the soluble protein in potato tubers. Mignery et al. Gene. 1988; 62(1):27-44. And, despite potato juice conventionally being considered waste, the nutritional quality of potato proteins is greater than that of casein and comparable to that of whole egg. Potato protein is rich in lysine and theoretically an excellent supplement for lysine-poor proteins such as those of cereals. Despite its unique nutritional qualities, potato protein is currently only used as animal feed, because the available products exhibit a number of serious drawbacks. Thus, patatin represents an attractive and abundant protein that can be used in various applications. However, isolation of patatin remains a challenge. There remains a need to effectively and efficiently isolate patatin from a sample, such as potato juice.

### SUMMARY

Disclosed herein are methods of isolating a protein from a sample using protein extraction and purification methods that enhance isolation of protein from a sample. Featured are methods that employ a sulfonated epoxy resin that increase recovery percentage of a protein (e.g., patatin) from a sample (e.g., potato juice) using reduced concentrations of NaOH (e.g., during wash and/or clean up steps) and more simplified post-purification steps such as elimination of Clean In Place (CIP) requirements compared to methods used in the art.

The disclosure herein demonstrates that a scalable, robust and cost-effective sulfonated epoxy resin can operate at a lower pH range (4.0-4.5) compared to other resins (e.g., a weak acid cation resin, which optimally operates at a higher pH range (5.2-6.0)). Different from other resins on the market, the porosity is also adjusted to accommodate large proteins that are found in various vegetable juices and dairy waste streams. In addition, because the resins utilized in the methods disclosed herein operate at a lower pH range, other food proteins such as those isolated from alcohol products such as beer, certain vegetables, and dairy products can be isolated using the methods disclosed herein. The present disclosure provides methods that isolate proteins of interest such as patatin while separating other proteins such as low molecular weight (LMW) proteins in a sample such as potato juice.

The sulfonated epoxy resin used in the disclosure provides a higher protein capacity, quicker protein extraction time, and lower eluent volume compared to other resins (e.g., a weak acid cation resin). Further, the methods provided herein allow for elution of both high and low molecular weight proteins, thus eliminating the need for a flow-through step and resulting in purer protein extracts compared to methods using other resins (e.g., a weak acid cation resin). Finally, the resin used in the methods disclosed herein can be regenerated for multiple uses.

In one instance, featured herein is a method of isolating a protein fraction from a potato sample comprising (a) adjusting the potato sample to a pH of about 4.0 to 5.2; (b) loading the potato sample onto a sulfonated epoxy resin, wherein the sulfonated epoxy resin is at a pH of about 4.0 to 4.5 and wherein a sulfonated functional group is covalently bound to the sulfonated epoxy resin; wherein the sulfonated epoxy resin comprises Formula (I):

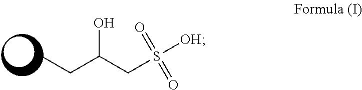

(c) washing the sulfonated epoxy resin; and (d) eluting the protein fraction in a solution comprising a pH of about 6.0. In some instances, the protein fraction is patatin.

In a second instance, also featured herein is a method of isolating a protein fraction from a sample comprising (a) adjusting the sample to a pH of about 4.0 to 5.2; (b) loading the potato sample onto a sulfonated epoxy resin, wherein the sulfonated epoxy resin is at a pH of about 4.0 to 4.5 and wherein a sulfonated functional group is covalently bound to the sulfonated epoxy resin; wherein the sulfonated epoxy resin comprises Formula (I):

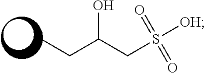

(c) washing the sulfonated epoxy resin; and (d) eluting the protein fraction in a solution comprising a pH of about 6.0; wherein the sample is selected from a fermented sample, soy, or a dairy product.

In some instances, the fermented sample is a beer. In some instances, the sample is a frozen sample. In some instances, the sample is a fresh sample. In some instances, the pH of the sample initially is from 5.2-6.0. In some instances, the pore diameter of the sulfonated epoxy resin is about 1000 Angstroms. In some instances, the sulfonated epoxy resin has a capacity of about 40 mg/ml. In some instances, the sulfonated epoxy resin has a maximum working pressure of about 20 bar. In some instances, the sulfonated epoxy resin is capable of purifying proteins up to 250 kDa.

In some instances, the wash step utilizes a reduced amount of sodium hydroxide (NaOH) compared to a wash step that uses a weak acid cation ion exchange resin. In some instances, the method utilizes fewer post elution purification steps compared to a method that uses a weak acid cation ion exchange resin. In some instances, the method utilizes fewer Clean In Place (CIP) requirements steps compared to a method that uses a weak acid cation ion exchange resin. In some instances, the method results in an increased recovery percentage of patatin compared to a method that uses a weak acid cation ion exchange resin. In some instances, the sulfonated epoxy resin is calibrated prior to the step of loading the sample onto the sulfonated epoxy resin. In some instances, the sample is centrifuged prior to loading.

In some instances, the percentage of the protein fraction in the sample is about 1.2% to 2.0%. In some instances, the adjusting the sample uses hydrogen chloride (HCl). In some instances, the HCl is at a concentration of about 1 molar. In some instances, the sample is filtered prior to loading. In some instances, the sample is filtered through a grade 1 Whatman filter. In some instances, the sample is washed with a first wash buffer. In some instances, the sample is washed with a second wash buffer. In some instances, the protein fraction is eluted using a first elution buffer. In some instances, the sample further comprises low molecular weight (LMW) proteins, and wherein the LMW proteins are eluted using a second elution buffer.

In some instances, the methods disclosed herein further comprise measuring the absorption of diluted iTAG at a wavelength of 482 nm (Sprint method).

In some instances, the protein fraction is visualized on an SDS-PAGE gel.

In some instances, the sulfonated epoxy resin is used more than one time.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows SDS-PAGE gel images showing the proteins present in potato juice from Supplier 1 and Supplier 2. Bands at ca. 88 kDa (dimer) and ca. 37 kDa represent patatin, <25 kDa represent LMW proteins. MW—Molecular Weight, kDa—kiloDaltons.

FIG. 2 shows an SDS-Page gel image showing detection of patatin around 37 kDa: in potato juice, in a sample of potato juice that has been applied to a weak acid cation resin at pH 5.2 (“Load”), in a wash sample from a weak acid cation resin that has been loaded with potato juice at pH 5.2 (“Wash”), and in a sample that has been eluted with sodium hydroxide from a weak acid cation resin that has been loaded with potato juice at pH 5.2 (“Elute 1”). Bands lower than 20 kDa are low molecular weight (LMW) proteins and bands of approx. 88 kDa are patatin dimer.

FIG. 3 shows an SDS-Page gel image showing detection of patatin around 37 kDa: in potato juice, in a sample of potato juice that has been applied to a sulfonated epoxy resin at pH 4.0 (“Load”), in a wash sample from a sulfonated epoxy resin that has been loaded with potato juice at pH 4.0 (“Wash”), in a sample that has been eluted by pH adjustment to 6.0 from a sulfonated epoxy resin that has been loaded with potato juice at pH 4.0 (“Elute 1”) and in a sample that has been eluted with sodium hydroxide from a sulfonated epoxy resin that has been loaded with potato juice at pH 4.0 (“Elute 2”). Bands lower than 20 kDa are low molecular weight (LMW) proteins and bands of approx. 88 kDa are patatin dimer.

FIG. 4 shows an SDS-Page gel image showing detection of proteins present in a sample of beer before and after treatment using various resins. MQ/M: Chromalite® methacrylic SBA (quaternary ammonium) resin; MS/M: a Chromalite® methacrylic SAC (sulfonic acid) resin; MCM/M: a Chromalite® methacrylic (WAC) (carboxylic acid) resin; MDEA/M: a Chromalite® methacrylic WBA (diethylamine) resin; D6373: a Chromalite® methacrylic/PVP copolymer resin; Becosorb 2500: a silica resin.

Patatin represents a group of glycoprotein isoforms with molecular mass 40-44 kDa and its appearance on an SDS-PAGE gel is spread across this molecular weight range. Additionally, the presence of protease enzymes within the low molecular weight fraction can result in the appearance at lower than expected MW (35-44 kDa), most likely due to proteolytic cleavage, a well-known effect observed in the analysis if protein mixtures by SDS-Page techniques.

### DETAILED DESCRIPTION

The present disclosure relates to methods for the isolation of a protein fraction from a sample. Specifically, a method for the fractionation of proteins at a low pH (4.0-4.5) is provided, allowing for the separation and isolation of a high molecular weight fraction and a low molecular weight fraction. In some instances, the protein fraction is patatin and the sample is potato juice.

### Methods of Chromatographic Protein Extraction and Purification

Disclosed herein are methods of protein extraction and purification of a protein fraction (e.g., any of the protein fractions disclosed herein; e.g., HMW protein; e.g., patatin) from a sample (e.g., any of the samples disclosed herein; e.g., potato juice). At the outset, in some instances, a potato protein fraction is obtained by loading a potato sample onto a sulfonated epoxy resin which results in the selective absorption of the HMW-fraction of potato protein, resulting in HMW-depleted potato juice. In some instances, the sample is equilibrated. Equilibration includes adjusting the pH of the potato juice or of the resin. Subsequently, the potato juice is contacted with (i.e., adsorbed to) a resin as described herein. In some instances, this step results in the selective absorption of the HMW-fraction of potato protein, resulting in LMW-depleted potato juice. Then, the methods disclosed herein include elution of the HMW-fraction (e.g., containing patatin).

At any point before or after any of the steps for the methods disclosed herein, the resin can be washed using any method known in the art. For example, in some instances, the resin can be rinsed using deionized (DI) water. In some instances, the resin can be washed using sodium hydroxide (NaOH). In some instances, the concentration of sodium hydroxide can be adjusted according to methods known by those skilled in the art. In some instances, the wash buffer is sodium citrate. In some instances, the resin is prepared by rinsing with sodium chloride (NaCl).

Where the pH of the potato juice requires adjustment during processing, such as for instance to a lower pH, or to a higher pH, acid or base is used to adjust the pH. Such acids and bases for pH-adjustment of potato juice may advantageously be weak acids, such as for instance citric, lactic, formic, acetic or phosphoric acid, or strong acids like nitric acid, hydrochloric and/or sulfuric acid. Also, it may be possible that during processing of potato juice adjustment to higher pH is required. In some instances, strong bases include, for instance sodium hydroxide, potassium hydroxide or calcium hydroxide. In some instances, weak bases include for instance, sodium and potassium salts of carbonate, phosphate, acetate, citrate and (bi-)sulfite. When buffers are used, combinations of above-mentioned or other weak acids and weak bases as known in the art are used to adjust the potato juice to the required pH.

In some instances, the methods include preparation of a sample. Preparation of the sample includes but is not limited to diluting the sample to a desired concentration. In some instances, to achieve a desired, pre-adsorption concentration, a sample (or diluted sample) can be measured at a particular wavelength (e.g., 482 nm). In some instances, the conductivity of the sample is measured prior to loading the sample onto the resin. In some instances, the pH of the sample is measured and can be adjusted using methods, acids or bases described above.

In some instances, the sample is pretreated to adjust the pH of the sample. In some instances, the sample is treated to achieve a pH of about 4.0 to 5.2. In some instances, the sample is treated to achieve a pH of about 4.0, about 4.1, about 4.2, about 4.3, about 4.4, about 4.5, about 4.6, about 4.7, about 4.8, about 4.9, about 5.0, about 5.1, or about 5.2.

In some instances, the methods include preparation of a resin. In some instances, preparation of the resin includes packing the column using methods known in the art. In some instances, the resin is prepared by rinsing with DI water. In some instances, the resin is prepared by rinsing with NaCl.

In one aspect of the disclosure, multiple resins are used. In some instances, the multiple resins include one sulfonated epoxy resin and one weak acid cation ion exchange resin using tandem purification methods.

Weak anion cation exchange resins are disclosed in US-2010-0040591-A1, US-2015-0183840-A1, and US-2019-0248842-A1, each of which are incorporated by reference in its entirety.

In some instances, the methods include loading of the prepared sample onto the resin. In some instances, the prepared sample is diluted in a loading buffer. As used herein, the loading buffer includes the composition comprising the protein of interest and one or more contaminants. The loading buffer has a conductivity and/or pH such that the protein of interest (and generally one or more contaminants) is/are bound to the resin.

In some instances, the methods include eluting a protein fraction from the resin using an elution buffer. As used herein, an elution buffer includes a buffer that is used to elute the protein of interest from the resin. The conductivity and/or pH of the elution buffer is/are such that the polypeptide of interest is eluted from the resin.

In some instances, the methods include evaluating the relative size and amounts of proteins after each step. In some instances, the methods include measuring the amount (e.g., concentration) of the protein of interest in the eluate. In some instances, a sample from each step is run on an SDS-PAGE gel and imaged to determine the fractions of proteins present at each step.

In some instances, the resin can be regenerated. Regeneration is a process that takes ion exchange resin beads that are exhausted (fully loaded), and removes ions that have been picked up during the in-service cycle so the resin can continue to be used. In some instances, the resin can be regenerated multiple times. In some instances, the resin is regenerated by a high concentration (10% brine) of salt or other regenerant chemical to restore the resin's capacity. In some instances, the sample is regenerated using NaOH. In some instances, when a resin disclosed herein is regenerated, about 60%-100% of the total ion exchange resin capacity is restored. In some instances, when a resin disclosed herein is regenerated, about 60%-80% of the total ion exchange resin capacity is restored. In some instances, when a resin disclosed herein is regenerated, about 60%, about 65%, about 70%, about 75%, about 80%, about 85%, about 90%, about 95%, or about 100% of the total ion exchange resin capacity is restored.

In some instances, after a protein fraction is isolated, the protein isolates are ultrafiltrated at pH values of 4.0-8.0, preferably pH 6.0-7.5. For protease inhibitor isolates pH values of 3-7, preferably 3.2-4.5 are used. In some instances, after removal of impurities, the pH is increased to pH 7-10 to enable high fluxes through the membranes. In some instances, protease inhibitors are processed at low pH of 3.0-5.0.

Apart from the purification steps, the native potato protein isolates obtained by the process of the disclosure may be concentrated up to more than 20% dry matter by evaporation, freeze concentration, or isoelectric precipitation using carbon dioxide. The dry matter of these concentrates can contain more than 85% of protein, preferably more than 90% of protein. The dried products can contain more than 90%, preferably more than 92% of protein, with a moisture level of 4-9%.

### Samples and Resins

In some instances, the sample used in the methods disclosed herein is a biological sample. In some instances, the sample is a vegetable. Suitable vegetables include but are not limited to potatoes, watercress, alfalfa sprouts, spinach, cabbage, asparagus, mustard greens, broccoli, broccoli rabe, collard greens, eggplant, carrots, peas, red bell peppers, kale, zucchini, cauliflower, Brussel sprouts, sweet potatoes, corn, or squash. In some instances, the sample is a potato.

In some instances, the sample is a vegetable fraction that is isolated from any one of the vegetables listed herein. In some instances, the sample is a byproduct of potato starch production. In some instances, the sample is potato juice. In some instances, the potato juice is a byproduct of potato starch production. For the present disclosure, potato juice is understood to mean any kind of potato-derived liquid that contains a significant amount of native potato proteins, and includes among others potato fruit juice (PFJ) that is normally obtained as a by-product of starch production, as well as diluted PFJ known as potato fruit water (PFW). Thus, it can be used in the form in which it is generally considered a waste stream, for instance from the production of potato starch, or from the processing of consumption potatoes, both in diluted or in partially processed form.

Potato juice can be stored prior to protein fractionation. In some instances, the potato juice is stored at −20° C. prior to protein fractionation. In some instances, the potato juice is stored at −80° C. prior to protein fractionation.

Potato juice can also be depleted of a particular fraction of potato proteins, such as for example obtained after application of a process according to the present invention. For example, potato juice that has been cleared or partially cleared of the protease inhibitor fraction, referred to as the low molecular weight (LMW)-fraction, is called LMW-depleted or partially LMW-depleted potato juice. Also, potato juice that has been cleared or partially cleared of the patatin fraction, referred to as the high molecular weight (HMW)-fraction, is called HMW-depleted or partially HMW-depleted potato juice. In some instances, the high molecular weight (HMW) is the patatin rich fraction of potato proteins. In some instances, the low molecular weight (LMW)-fraction is rich in protease inhibitors.

In some instances, the potato juice is crude potato juice. Crude potato juice is potato juice that has not undergone any purification and can be used as such, or first be subjected to a non-invasive pre-treatment that does not affect the content and nature of the potato proteins included in the juice. Such treatments may for instance be pH-adjustment, clarification or color removal, such as done by centrifugation, filtration, flocculation and/or absorption. Specifically, various processes exist for pre-cleaning potato juice, with the aim of removing for instance unwanted contaminants, such as for instance glycoalkaloids, (poly)phenols, pectins, lipids, fatty acids and/or proanthocyanidines and colored derivatives thereof, such as epicatechins and anthocyanines. Processes for removing one or more of these contaminants, specifically glycoalkaloids, are described in pending applications WO 2008/056977, which is incorporated by reference in its entirety and which discloses absorption of contaminants from a potato juice process stream by contact with a layered silicate. Alternatively, WO 2008/069651, which is incorporated by reference in its entirety, discloses absorption of contaminants from an aqueous solution of a vegetable process stream by contact with activated carbon. A process comprising pre-treatment of the potato juice before application of the process of the present invention may be especially advantageous when using a pre-treatment comprising a flocculation process, for example with bivalent metal ions such as calcium or magnesium salts.

In some instances, the sample includes a plurality of proteins. In some instances, the plurality of proteins includes a protein of interest that is purified using methods disclosed herein. In some instances, the protein of interest is patatin. Patatin is a 44 kiloDalton (kDa) protein that functions as a storage protein in a potato. In some instances, patatin is isolated and purified using methods disclosed herein. In some instances, the sample includes proteins other than one or more proteins of interest. In some instances, proteins other than one or more proteins of interest include low molecular weight (LMW) proteins, including protease inhibitors and other small proteins (5-25 kDa).

In some instances, the sample is an alcohol sample. In some instances, the alcohol sample is a beer sample. In some instances, protein is isolated and purified from the beer sample.

In some instances, the sample is a dairy product. In some instances, the sample is milk. In some instances, the protein isolated from milk using methods disclosed herein include lactoferrin.

In some instances, disclosed herein are methods of isolating proteins of interest using chromatographic protein extraction and purification methods that utilize a resin. In some instances, the resins used in the methods disclosed herein are hydrophilic, macroporous, methacrylic resins for large-scale applications. In some instances, the resin has a rigid polymeric backbone that ensures consistent pressure-flow properties. In some instances, the resins disclosed herein is a strong cation exchanger.

In some instances, the resin (shown as a sphere in Formulae (I) and (III) below) includes a ligand. In some instances, the ligand is a sulfopropyl functional group. In some instances, the sulfopropyl functional group is shown in Formula (I) below.

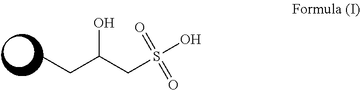

In some instances, the sulfopropyl functional group is shown in Formula (III) below.

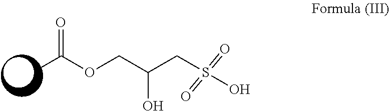

In some instances, the pKa of the ligand should preferably be between 2.5-5, more preferably between 4-5. Ligands may be aliphatic, substituted aliphatic or aromatic ligands, including substituted and/or heteroaromatic groups, or combinations thereof. Suitable aromatic groups are for instance substituted or unsubstituted phenyl, naphtyl, pyridine and pyrimidine structures. In some instances, the ligand is based on an alkyl chain, with length of about 2-8 carbon atoms. In some instances, the alkyl chain has 2 carbon atoms. In some instances, the alkyl chain has 3 carbon atoms. In some instances, the alkyl chain has 4 carbon atoms. In some instances, the alkyl chain has 5 carbon atoms. In some instances, the alkyl chain has 6 carbon atoms. In some instances, the alkyl chain has 7 carbon atoms. In some instances, the alkyl chain has 8 carbon atoms. In some instances, the ligand includes a functional group. Without limitation, examples of functional groups include sulfonic groups (e.g., sulfonic acid), amino acids, carboxylic acid, thiol, purine, phenol, pyrazine, sulfoxide, thiazole, amine, imidazole, piperazine functional groups. In some instances, the functional group is a charged functional groups that includes, but are not limited to, sulfonic acid, phosphonic acid, carboxylic acid, and amines, and their corresponding ions: sulfonate, phosphonate, carboxylate, and ammonium. In some instances, the functional group includes polyacrylic acid (PAA) and polyvinyl sulfonic acid (PVS), and their anions, polyacrylate and polyvinyl sulfonate.

In some instances, the functional group includes one or more additional cationic and anionic charged functional groups. Additional exemplary functional groups include polyacrylic acid (PAA), polyvinyl sulfonate (PVS), polystyrene sulfonic acid (PSS, poly (4-vinylbenzenesulfonate, metal salt), polymethacrylate (PMA), polyacrylamide methylpropanesulfonate (PAMPS), CMP (PAMPS) maleic anhydride-styrene copolymer (MAS), a maleic anhydride-vinyl methyl ether copolymer (MAVE), polyaspartate, polyglutamate, dextransulfate, pectin, alginate and glycosaminoglycans such as chondroitin sulfate, heparin/heparana acid; and all their salts and copolymers.

In some instances, the functional group is a sulfonic functional group. In some instances, the functional group includes one or more of a modifier including, but are not limited to, zwitterion, ampholyte, amino acid, aminoalkyl sulfonic acid, aminoalkyl carboxylic acid, mono and dimethylaminoalkyl sulfonic acid, mono and dimethylaminoalkyl carboxylic acid, pyridinium alkyl Also included are sulfonic acids, as well as pyridinium alkyl carboxylic acid groups. Alternatively, amphoteric charge ionic modifiers include 2-(N-morpholino) ethanesulfonic acid, 3-(N-morpholino) propanesulfonic acid, 4-(2-hydroxyethyl)-1-piperazine ethanesulfonic acid, piperazine-N, N′-bis (2-ethanesulfonic acid), N-cyclohexyl-3-aminopropanesulfonic acid, N-cyclohexyl-2-hydroxyl-3-aminopropanesulfonic acid, 3-[(3-colamidopropyl) Dimethylammonio]-1-propanesulfonate, 6-methyl-9,10-didehydroergoline-8-carboxylic acid, phenolsulfophthalein, betaine, quinonoid, N, N-bis (2-hydroxyethyl) Also included are glycine and N-[tris (hydroxymethyl) methyl] glycine groups.

In some instances, the functional group includes an epoxy group. In some instances, the resin is an epoxy methacrylate macroporous affinity resin. In some instances, the resin comprising an epoxy group includes a rigid polymeric backbone, thereby providing increased stability during purification steps (e.g., NaOH washes). In some instances, the resin comprising an epoxy group is stable over a wide range of pH (e.g., pH 2-12 range for normal operating conditions and pH 1-13 for cleaning conditions). In some instances, the resin has a matrix comprising methacrylate. In some instances, the resin has a matrix comprising styrene. In some instances, the resin has a matrix comprising agarose.

In some instances, the functional group is a sulfopropyl/Na+ group. In some instances, the functional group is a carboxymethyl/Na+ group. In some instances, the functional group is a quaternary ammonium/Cl− group. In some instances, the functional group is a diethylamine group.

In some instances, the ligand that binds a target protein is selected so as to obtain that the ligand, under the conditions permitting the target protein to bind to the resin material, can be electrostatically charged or uncharged depending on the buffer pH. Prior to loading the aqueous medium onto the column, the resin material is typically equilibrated to the pH conditions at which the binding of the target protein occurs. It has been found that the resin materials of the invention are highly effective even at a relatively low density. In preferred embodiments, the process of the invention recovers at least 50% of the total amount of target protein initially present in the aqueous medium, including at least 60%, 10 70%, 80% or 90% recovery. Even higher recovery rates have been found, such as at least 92.5%, 95%, 97.5% or at least 99%.

In some instances, the resin disclosed herein include particles that have a plurality of beads. In some instances, the radius of a bead in the plurality of beads ranges from about 30 μm to about 500 μm. In some instances, the radius of the bead in the plurality of beads ranges from about 30 μm to about 500 μm. In some instances, the radius of the bead in the plurality of beads ranges from about 30 μm to about 500 μm, from about 30 μm to about 400 μm, from about 30 μm to about 300 μm, from about 30 μm to about 200 μm, or from about 100 μm to about 75 μm. In some instances, the radius of a bead in the plurality of beads is about 30, 40, 50, 60, 70, 80, 90, 100, 150, 200, 250, 300, 350, 400, 450, or 500 μm. In some instances, the radius of a bead in the plurality of beads ranges from about 35 μm to about 65 μm In some instances, the radius of a bead in the plurality of beads ranges from about 40 μm to about 60 μm. In some instances, the radius of a bead in the plurality of beads ranges from about 37.5 μm to about 62.5 μm. In some instances, the radius of a bead in the plurality of beads is about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 μm. In some instances, the particles have a bead size of about 37.5 μm. In some instances, the particles have a bead size of about 62.5 μm.

In some instances, the adsorbent carrier ligand for adsorption for biomolecules for the resins disclosed herein is in a concentration in the range of 100-200 mM. In some instances, the concentration is about 100, about 110, about 120, about 130, about 140, about 150, about 160, about 170, about 180, about 190, or about 200 mM.

In some instances, the resins disclosed herein promote a simpler chemistry compared to other resins (e.g., weak acid cation resins). In some instances, the resins disclosed herein include one or more methacrylates. Methacrylic resins are disclosed in U.S. Pat. No. 7,964,690 B2, which is incorporated by reference in its entirety. In some instances, the resins disclosed herein consist only of one or more methacrylates. In some instances, the resins disclosed herein do not include benzyl. In some instances, the resins disclosed herein do not include phenyl. In some instances, the resins disclosed herein do not include benzyl or phenyl.

In some instances, the resins disclosed herein are hydrophilic. In some instances, the resins disclosed herein include hydrophilic macroporous methacrylic resins, for the chromatographic purification of peptides, oligonucleotides, proteins, enzymes and biomolecules. In some instances, the ligand in the resin is not hydrophobic. In some instances, the ligand in the resin in hydrophilic.

Further disclosure of resins is disclosed in WO-2014011042, WO-2001058924-A2, US-20070092960-A, and WO-20150183840-A1, each of which are incorporated by reference in its entirety.

In some instances, the resin that includes Formula (I) purifies a protein fragment at a size up to 250 kDa. In some instances, the resin that includes Formula (I) purifies a protein fragment at a size of 20 to 250 kDa. In some instances, the resin that includes Formula (I) purifies a protein fragment at a size of 20 to 200 kDa. In some instances, the resin that includes Formula (I) purifies a protein fragment at a size of 20 to 150 kDa. In some instances, the resin that includes Formula (I) purifies a protein fragment at a size of 20 to 100 kDa. In some instances, the resin that includes Formula (I) purifies a protein fragment at a size of 20 to 50 kDa. In some instances, the resin that includes Formula (I) purifies a protein fragment at a size of 37 kDa.

In some instances, the resin has the capacity to filter various volumes of a sample. In some instances, the resin filters 25 ml of a sample. In some instances, the resin filters 100 ml of a sample. In some instances, the resin filters 500 ml of a sample. In some instances, the resin filters 1 L of a sample. In some instances, the resin filters 5 L of a sample. In some instances, the resin filters 10 L of a sample.

The total capacity for a resin is a representation of the total number of exchange sites built into the resin. In some instances, the resin disclosed herein has a total capacity (min.) of about 20 mg/ml. In some instances, the resin disclosed herein has a total capacity (min.) of about 30 mg/ml. In some instances, the resin disclosed herein has a total capacity (min.) of about 40 mg/ml. In some instances, the resin disclosed herein has a total capacity (min.) of about 50 mg/ml. In some instances, the resin disclosed herein has a total capacity (min.) of about 60 mg/ml. In some instances, the resin disclosed herein has a total capacity (min.) of about 70 mg/ml. In some instances, the resin disclosed herein has a total capacity (min.) of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 mg/ml.

In some instances, the resin disclosed herein has a capacity (min.) for a high molecular weight (HMW) fraction of total potato protein of about 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg/ml. In some instances, the resin disclosed herein has a capacity (min.) for a low molecular weight (LMW) fraction of total potato protein of about 40, 41, 42, 43, 44, 45, 46, 47, 48, 49, 50, 51, 52, 53, 54, 55, 56, 57, 58, 59, or 60 mg/ml.

In some instances, the resin has a working pressure (max.) of about 1 bar to 10 bar, about 2 bar to 10 bar, about 3 bar to 10 bar, about 4 bar to 10 bar, about 5 bar to 10 bar, about 6 bar to 10 bar, about 7 bar to 10 bar, about 8 bar to 10 bar, or about 9 bar to 10 bar. In some instances, the resin has a working pressure (max.) of about 5 bar to 10 bar. In some instances, the resin has a working pressure (max.) of about 10 bar to 40 bar. In some instances, the resin has a working pressure (max.) of about 15 bar to 35 bar. In some instances, the resin has a working pressure (max.) of about 15 bar to 25 bar. In some instances, the resin has a working pressure (max.) of about 10 bar. In some instances, the resin has a working pressure (max.) of about 20 bar. In some instances, the resin has a working pressure (max.) of about 30 bar. In some instances, the resin has a working pressure (max.) of about 40 bar. In some instances, the resin has a working pressure (max.) of about 1 bar to about 20 bar, about 2 bar to about 20 bar, about 3 bar to about 20 bar, about 4 bar to about 20 bar, about 5 bar to about 20 bar, about 6 bar to about 20 bar, about 7 bar to about 20 bar, about 8 bar to about 20 bar, about 9 bar to about 20 bar, about 10 bar to about 20 bar, about 11 bar to about 20 bar, about 12 bar to about 20 bar, about 13 bar to about 20 bar, about 14 bar to about 20 bar, about 15 bar to about 20 bar, about 16 bar to about 20 bar, about 17 bar to about 20 bar, about 18 bar to about 20 bar, or about 19 bar to about 20 bar.

The resin disclosed herein can be used for small-scale analysis (e.g., in the setting of analyzing small particles) to large-scale chromatography. In some instances, the resin has a pore diameter that ranges from 100 Å to 2000 Å. In some instances, the resin has a pore diameter that ranges from 500 Å to 1500 Å. In some instances, the resin has a pore diameter that ranges from 750 Å to 1250 Å. In some instances, the resin has a resin pore diameter of about 100 Å, about 200 Å, about 300 Å, about 400 Å, about 500 Å, about 600 Å, about 700 Å, about 800 Å, about 900 Å, about 1000 Å, about 1100 Å, about 1200 Å, about 1300 Å, about 1400 Å, about 1500 Å, about 1600 Å, about 1700 Å, about 1800 Å, about 1900 Å, or about 2000 Å. In some instances, the pore diameter is about 30-220 nm. In some instances, the pore diameter is about 150-200 nm. In some instances, the pore diameter is about 30, 40, 50, 60, 70, 80, 90, 100, 110, 120, 130, 140, 150, 160, 170, 180, 190, 200, 210, or 220 nm.

In some instances, the resin is stable at various pH levels. In some instances, the resin disclosed herein is stable at pH at a range of about 1-14, about 2-13, about 3-13, about 4-11, about 4-10, about 4-9, about 4-8, about 4-7, about 4-6, or about 4-5. In some instances, the resin disclosed herein is stable at pH of about 1, about 2, about 3, about 4, about 5, about 6, about 7, about 8, about 9, about 10, about 11, about 12, about 13, about 14. In some instances, the resin disclosed herein is stable at pH of about 4. In some instances, the resin disclosed herein is stable at pH of about 5.

In some instances, the resin includes a sulfonated epoxy functional group as shown in Formula (I), has a pore diameter of about 1000 Å; has a working pressure (max.) of about 20 bar; and has a total capacity (min.) of about 40 mg/ml. In some instances, the resin disclosed herein is capable of filtering a protein at a particle size of 100-300 μm.

### Methods of Preparing Resins

Also provided herein are methods of preparing a resin described herein. In some instances, the methods include providing a mixture comprising Formula (I).

In some instances, the sulfonated resin is activated. In some instances, the resin includes beads. In some instances, the beads are epoxy beads. In some instances, the epoxy beads are functionalized to add a sulfonated group, thereby manufacturing a sulfonated resin, as shown below.

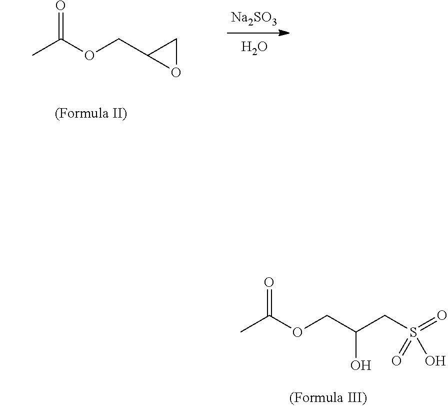

In some instances, the resin manufactured herein includes Formula III.

As shown above, the epoxy-functionalized beads are functionalized to manufacture a sulfonated resin. In some instances, Na₂SO₃ is used as a source of SO₃ for the functionalization of the free epoxides of the epoxy copolymer. Other sources of SO₃ can be used, including but not limited to Li₂SO₃, K₂SO₃, and Rb₂SO₃. In some instances, the reaction includes a nucleophilic addition of sulphite to the free epoxide groups on the copolymer structure. In some instances, the methods include heating of the resin in a solution of Na₂SO₃. In some instances, the resin is heated at about 50° C., about 60° C., about 70° C., about 80° C., or about 90° C. In some instances, the resin is heated at about 80° C. In some instances, the resin is heated at a temperature disclosed herein for about 1 hour, for about 2 hours, for about 3 hours, for about 4 hours, or four about 5 hours. In some instances, the resin is heated for about 4 hours. In some instances, the resin is heated in a solution of Na₂SO₃ for 4 hours at 80° C. It is noted that the concentration of the Na₂SO₃ solution during the reaction is important to ensure consistent functionalisation of the resin. Using the dry weight of the resin it is possible to reduce batch to batch variation in the overall concentration of Na₂SO₃, which in turn affects the functionalization.

Next the resin is quenched with an aqueous solution. In some instances the resin is quenched with aqueous HCl. In some instances, after quenching, the batch and column are washed using any wash solution known in the art.

In some instances, the mixture to manufacture the resin includes epichlorohydrin, epoxy methacrtlate, or a compound that can be used for the production of the epoxy resin. In some instances, the process of the present disclosure provide preparing a sulfonated epoxy resin using stoichiometry which results in unreacted epoxide groups. In some instances, the epoxy resin is an epoxide-containing compound having an average of more than one epoxide group per molecule. Additional disclosure of preparation of epoxy-sulfonated resins is disclosed in WO-2018-005307-A1, US-2019-0256644-A1, and U.S. Pat. No. 4,446,257, each of which is incorporated by reference in its entirety.

In some instances, the reaction is performed at about room temperature. In some instanced, the reaction is performed at about 10° C., 15° C., 20° C., 25° C., 30° C., 35° C., 37° C., 40° C., 45° C., or 50° C. In some instances, the reaction is performed at pH 7.5-8.5. In some instances, the reaction is performed at about pH 9. In some instances, the reaction is performed at about pH 11-13. In some instances, a base is added to the mixture. In some instances, the base is sodium hydroxide or potassium hydroxide. In some instances, the purity of the Formula (I) in the resin can be determined using methods known in the art.

### EXAMPLES

### Example 1. Preparation of Sulfonated Epoxy Resin

To prepare the sulfonated epoxy resin, demineralized water was charged to a suitably sized reaction vessel and a pre-determined quantity of sodium sulfite was added portion-wise while stirring to dissolve. Epoxy functionalized polymer beads were slurried in demineralized water and added to the solution. The mixture was stirred for 20 minutes at room temperature before heating to 80° C. for 4 hours. The batch was cooled to room temperature and dilute HCl added in one portion followed by stirring. The mixture was filtered, and the activated beads washed with demineralized water three times, with dilute aqueous sodium hydroxide, followed by with demineralized water two times before being transferred to a column for final demineralized water rinsing (until pH of eluent approximate pH 7-8). The sulfonated epoxy beads were then removed from the column, and excess water was removed by filtration under vacuum conditions.

### Example 2. Preparation of Sample

Potato starch was generated and potato juice was manufactured as a byproduct. The pH of the potato juice was measured and the samples were adjusted to pH adjustment to 5.2 (to run on weak acid cation ion exchange resin) or 4.0 (for use on sulfonated epoxy resin resin) using HCl. The protein content of each sample is listed in Table 1.

TABLE 1
pH and concentration of potato juice samples

| Potato Juice | | Necessary Pre- | Protein Content |
|---|---|---|---|
| Sample | Initial pH | treatment | after Pre-treatment |
| Sample 1 | 5.2 | pH adjustment to 5.2 | 1.2-1.4 % |
| Sample 2 | 6.0 | (to run on weak acid | 1.6-2.0 % |
| | | cation ion exchange | |
| | | resin) or 4.0 (for use | |
| | | on sulfonated epoxy | |
| | | resin). Centrifugation | |
| | | at 4,100 rcf for 20 | |
| | | minutes | |

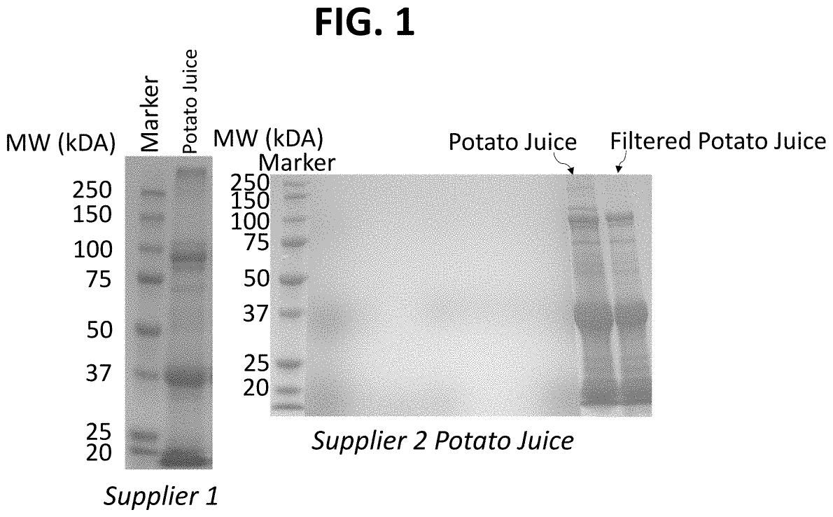

Loading of the potato juice to the column was controlled to give a consistent loading of 80 g protein per liter of resin. Filtered potato juice is the potato juice after centrifugation at 4,100 rcf for 20 minutes and filtered through 0.45 micron cellulose nitrate membrane. As shown in FIG. 1, a 37 kDa band was identified in both samples.

### Example 3. Comparison of Patatin Purification Using Different Resins

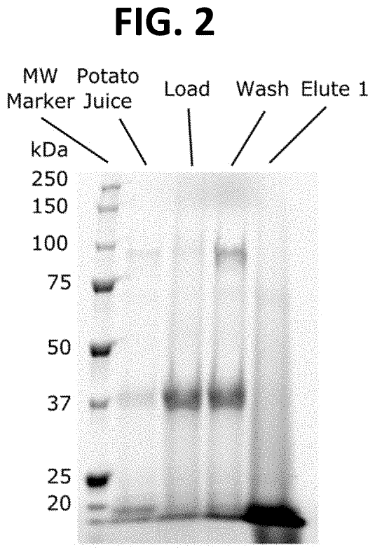

Patatin-containing samples were loaded onto a weak acid cation ion exchange resin or a sulfonated epoxy resin. Weak anion cation exchange resins are disclosed in US-2010-0040591-A1, US-2015-0183840-A1, and US-2019-0248842-A1, each of which are incorporated by reference in its entirety. Representative samples were isolated initially (i.e., potato juice), after loading, after washing the resin, and during two steps of elution. As shown in FIG. 2, three key protein components of potato juice were identified using the weak acid cation ion exchange resin at a pH of 5.2-6.0. High molecular weight proteins (ca. 100 kDa), patatin (44 kDa and minor signal ca. 88 kDa dimer), and protease inhibitors and other small proteins (ca. 5-25 kDa) are present in the samples before and after adsorption using a weak acid cation ion exchange resin. FIG. 2 also shows that the purification of potato juice using this method selectively removes the low molecular weight proteins from the sample, leaving patatin present in the load and wash fractions. Further, the LMW proteins are all located in the elute 1 fraction, as they have been bound to the resin and subsequently eluted with sodium hydroxide.

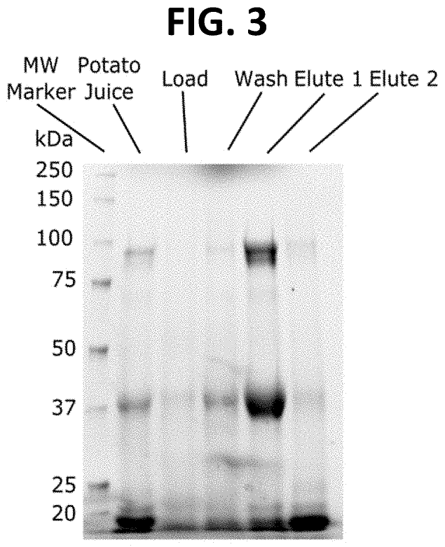

A similar experiment was run using a sulfonated epoxy resin at pH 4.0-4.5. As shown in FIG. 3, purification of potato juice using a sulfonated epoxy resin removes both the low molecular weight proteins and patatin from the sample, with elution occurring in two stages, first the patatin, followed by the LMW protein. The patatin is found in the elute 1 fraction, eluted using a pH change to 6.0 and the LMW proteins are all located in the elute 2 fraction, eluted with sodium hydroxide at a pH of 13 using 1M NaOH. As shown in FIG. 3, using the sulfonated epoxy resin at the lower pH range, significantly higher patatin purity is obtained. Further, there was no drop in capacity using the sulfonated epoxy resin, whereas there was a drop in capacity using the weak acid cation ion exchange resin (data not shown). In addition, because of the increased recovery, fewer downstream steps (e.g., post purification steps, reduced NaOH use, reduction/elimination of Clean In Place (CIP) requirements) are necessary. These data demonstrate that a sulfonated epoxy resin (as shown in FIG. 3) more effectively isolates patatin compared to a weak acid cation ion exchange resin (as shown in FIG. 2).

### Example 4. Use of a Sulfonated Epoxy Resin to Isolate Beer Protein

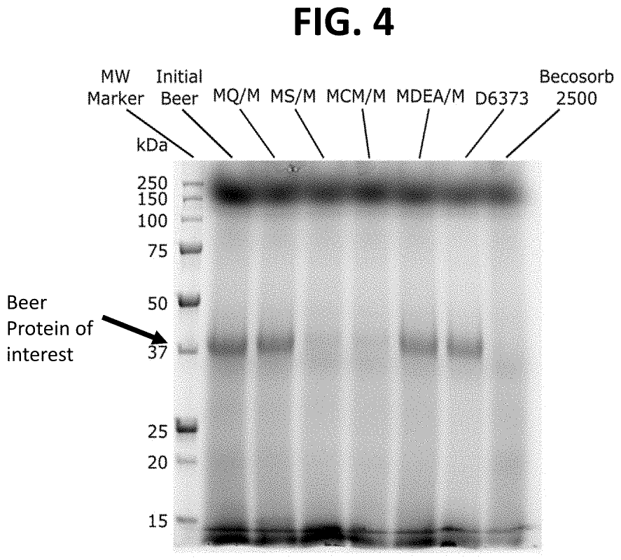

In order to test the efficacy of a sulfonated epoxy resin for isolation of other fragments, protein isolated from a beer was loaded onto various resins having different functional groups and capacities (shown in Table 2). Beer samples were provided by Handtmann Group sourced from a German brewery. Initial protein content of 6.5-7.0 g/l. Ethanol content 4.5% by volume. As shown in FIG. 4, the removal of a protein of molecular weight ca. 37 kDa was achieved at a pH of 4.5 by Chromalite MS/M and MCM/M. The total capacities of Chromalite M resins are shown in the below table for comparison.

TABLE 2
Capacity of resins in FIG. 4.

| Resin | Functional group | Capacity (g/L) |
|---|---|---|
| Chromalite ® Methacrylic | Quaternary Ammonium | 0.75 |
| strong base anion (SBA) | | |
| Chromalite ® Methacrylic | Sulfonic | 1.22 |
| strong anion (SA) | | |
| Chromalite ® Methacrylic | Carboxilic | 0.83 |
| weak acid cation (WAC) | | |
| Chromalite ® Methacrylic | Diethylamine | 1.17 |
| weak base anion (WBA) | | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of isolating a protein fraction from a potato sample comprising:
(a) adjusting the potato sample containing the protein fraction to a pH of about 4.0 to 5.2;
(b) loading the potato sample onto a sulfonated epoxy resin, wherein the sulfonated epoxy resin is adjusted to a pH of about 4.0 to 4.5 and wherein the sulfonated epoxy resin comprises Formula (I): 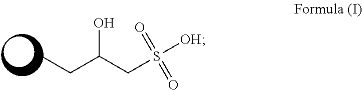
(c) washing the loaded sulfonated epoxy resin with a buffer; and
(d) eluting the protein fraction in a solution comprising a pH of about 6.0.

2. The method of claim 1, wherein the protein fraction is patatin.

3. A method of isolating a protein fraction from a sample comprising:
(a) adjusting the sample to a pH of about 4.0 to 5.2;
(b) loading the sample onto a sulfonated epoxy resin, wherein the sulfonated epoxy resin is at a pH of about 4.0 to 4.5 and wherein a sulfonated functional group is covalently bound to the sulfonated epoxy resin; wherein the sulfonated epoxy resin comprises Formula (I): 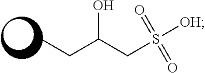
(c) washing the sulfonated epoxy resin; and
(d) eluting the protein fraction in a solution comprising a pH of about 6.0;
wherein the sample is selected from a fermented sample, soy, or a dairy product.

4. The method of claim 3, wherein the fermented sample is a beer.

5. The method of claim 1, wherein the sample is a frozen sample or a fresh sample.

6. (canceled)

7. The method of claim 1, wherein the pH of the sample initially is from 5.2-6.0.

8. The method of claim 1, wherein the pore diameter of the sulfonated epoxy resin comprises one or more of the following properties:
is about 1000 Angstroms;
has a capacity of about 40 mg/ml;
has a maximum working pressure of about 20 bar; or
is capable of purifying proteins up to 250 kDa.

9-11. (canceled)

12. The method of claim 1, wherein the wash step utilizes a reduced amount of sodium hydroxide (NaOH) compared to a wash step that uses a weak acid cation ion exchange resin.

13. The method of claim 1, wherein the method:
utilizes fewer post elution purification steps compared to a method that uses a weak acid cation ion exchange resin;
utilizes fewer Clean In Place (CIP) requirements steps compared to a method that uses a weak acid cation ion exchange resin; and/or
results in an increased recovery percentage of patatin compared to a method that uses a weak acid cation ion exchange resin.

14-15. (canceled)

16. The method of claim 1, wherein the sulfonated epoxy resin is calibrated prior to the step of loading the sample onto the sulfonated epoxy resin.

17. The method of claim 1, wherein the sample is centrifuged prior to loading.

18. The method of claim 1, wherein the percentage of the protein fraction in the sample is about 1.2% to 2.0%.

19. The method of claim 1, wherein the adjusting the sample uses hydrogen chloride (HCl).

20. (canceled)

21. The method of claim 1, wherein the sample is filtered prior to loading.

22. (canceled)

23. The method of claim 1, wherein the sample is washed with a first wash buffer and a second wash buffer.

24. (canceled)

25. The method of claim 1, wherein the protein fraction is eluted using a first elution buffer.

26. The method of claim 1, wherein the sample further comprises low molecular weight (LMW) proteins, and wherein the LMW proteins are eluted using a second elution buffer.

27. The method of claim 1, further comprising measuring the absorption of diluted iTAG at a wavelength of 482 nm (Sprint method).

29. The method of claim 1, wherein the sulfonated epoxy resin is used more than one time.

30. The method of claim 1, wherein the sulfonated epoxy resin comprises a matrix comprising methacrylate, styrene, or agarose.

31-32. (canceled)
